(19) United States
(12) Patent Application Publication
(10) Pub. No.: US 2013/0005722 A1
(43) Pub. Date: Jan. 3, 2013

(54) Pharmaceutical Composition of Mycophenolate Mofetil and Process for Preparing Thereof

(76) Inventors: Manoj Senapati, Orissa (IN); Manas Ranjan Pradhan, Orissa (IN); Vemula Sathyanarayana, Navi Mumbai (IN)

(21) Appl. No.: 13/583,387

(22) PCT Filed: Mar. 21, 2011
(86) PCT No.: PCT/IN2011/000186

§ 371 Date: Sep. 7, 2012

(30) Foreign Application Priority Data
Mar. 23, 2010 (IN) 783/MUM/2010

(51) Int. Cl. A61K 31/5377

(52) U.S. Cl. 514/233.5

(57) ABSTRACT

The present invention is a pharmaceutical composition comprising mycophenolate mofetil in an amount of about 75 to about 200 mg/ml and a suspending agent(s) in an amount of less than 1 mg/ml, as a dry formulation suitable, when constituted with water, for forming a suspension for oral administration, where mg/ml represents concentration after constitution with water.

### FIELD OF THE INVENTION

The present invention relates to pharmaceutical compositions of mycophenolate mofetil and processes for preparing thereof.

### BACKGROUND OF THE INVENTION

Mycophenolate mofetil (MMF) is an immunosuppressant and prodrug of mycophenolic acid used extensively in transplant medicine. The chemical name for mycophenolate mofetil is 2-morpholinoethyl (E)-6-(1,3-dihydro-4-hydroxy-6-methoxy-7-methyl-3-oxo-5-isobenzofuranyl)-4-methyl-4-hexenoate and so it is morpholinoethyl ester of mycophenolic acid. Its empirical formula is C₂₃H₃₁NO₇ and molecular weight 433.50. Its mode of action is as a reversible inhibitor of inosine monophosphate dehydrogenase (IMPDH) in purine biosynthesis. MMF is selective for the de novo pathway critical to lymphocytic proliferation and activation. Other cells are able to recover purines via a separate, scavenger pathway and are thus able to escape the effect. Mycophenolate mofetil is indicated for the prophylaxis of organ rejection in patients receiving allogeneic renal, cardiac or hepatic transplants. It should be used concomitantly with cyclosporine and corticosteroids. It is a useful alternative to azathioprine when Aza toxicity precludes use. In United States, it is marketed as CellCept® by Roche. CellCept® intravenous is an alternative dosage form to CellCept® capsules, tablets and oral suspension.

Mycophenolate mofetil requires daily doses on the order of 2.0 to as much as 3.5 or 4.0 grams per day depending upon the patient and the disease state being treated. Using a conventional dosage formulation containing 200 mg in a standard size 1 (0.48 cc volume) capsule, a patient receiving a 3.0 gram daily dose is required to take twelve capsules each day, giving rise to patient convenience and compliance concerns. As an alternative to this, oral suspensions are known for ease of administration, for example, to children or elderly adults, and mycophenolate mofetil oral suspensions are readily known in the art.

U.S. Pat. No. 4,753,935 relates to mycophenolate mofetil and specifically claims the compound. It also exemplifies oral suspension and its preparation but at a relatively low dose incorporating 1 gram of active compound in 100 ml.

U.S. Pat. No. 5,688,529A discloses high dose, dry granulations or powder blends and aqueous oral suspensions of mycophenolate mofetil or mycophenolic acid. It is disclosed that the dry suspension formulation contains active compound (7.5-30%), suspending/viscosity agent (1-30 mg/ml), sweetener, flavor, buffer (to a pH of 5-7.5), and optionally containing flavor enhancer, wetting agent, antimicrobial agent and color. According to the patent, xanthan gum, colloidal silicon dioxide, and sodium carboxymethyl cellulose are some of the suspending agents used. Though this patent discloses use of 1-30 mg/ml of the suspending agent, it discloses the suspending agents to range preferably from 10-30 mg/ml in its dry suspension formulation. The patent discloses sodium carboxymethyl cellulose to be used at 20 mg/ml and a combination of xanthan gum and colloidal silicon dioxide to be used at a range from 5.5-11.5 mg/ml of the dry suspension formulation, when reconstituted. The patent exemplifies the use of xanthan gum and colloidal silicon dioxide combination at 6 mg/ml and 11.5 mg/ml in different formulations, when reconstituted. The patent also exemplifies use of sodium carboxymethyl cellulose at 20 mg/ml in formulations, when reconstituted.

However, there still remains a need to provide commercially acceptable alternative dosage forms of mycophenolate mofetil oral suspensions. Surprisingly we have found that dry suspension formulation with high dose of mycophenolate mofetil can be prepared by using a suspending agent(s) in lesser amounts, than those used by the prior art formulations. It would always be desirable to have stable formulations which are equivalent to the branded composition but yet uses lesser amount of excipients.

We have found that the said compositions are having comparable physical stability as the commercially available compositions in the United States of America i.e. CellCept® suspensions, in spite of using lesser amounts of suspending agent(s).

We have also found that the said compositions are bioequivalent to the commercially available compositions in the United States of America i.e. CellCept® suspensions.

### OBJECT OF THE INVENTION

It is an object of the present invention to provide pharmaceutical compositions of mycophenolate mofetil and processes for preparing thereof.

It is an object of the present invention to provide a pharmaceutical composition comprising mycophenolate mofetil in an amount of about 75 to about 200 mg/ml and a suspending agent(s) in an amount of less than 1 mg/ml, as a dry formulation suitable, when constituted with water, for forming a suspension for oral administration, where mg/ml represents concentration after constitution with water.

It is also an object of the present invention to provide a process to prepare a pharmaceutical composition comprising mycophenolate mofetil in an amount of about 75 to about 200 mg/ml and a suspending agent(s) in an amount of less than 1 mg/ml, as a dry formulation suitable, when constituted with water, for forming a suspension for oral administration, where mg/ml represents concentration after constitution with water.

### SUMMARY OF THE INVENTION

The present invention relates to pharmaceutical compositions of mycophenolate mofetil and processes for preparing thereof.

The invention may be summarized as given below:

(A) A pharmaceutical composition comprising mycophenolate mofetil in an amount of about 75 to about 200 mg/ml and a suspending agent(s) in an amount of less than 1 mg/ml, as a dry formulation suitable, when constituted with water, for forming a suspension for oral administration, where mg/ml represents concentration after constitution with water.

(B) The pharmaceutical composition as in A above, comprising mycophenolate mofetil in an amount of about 75 to about 200 mg/ml and the suspending agent(s) in an amount of less than 1 mg/ml, provided in a container marked to be filled with purified water ranging from about 90 ml to about 400 ml to obtain a final predetermined volume ranging from about 175 ml to about 470 ml.

(C) The pharmaceutical composition as in A above, wherein the suspending agent(s) are selected from xanthan gum, hydroxy-propyl methylcellulose, microcrystalline cellulose, colloidal silicon dioxide, sodium carboxymethylcellulose and the like or mixtures thereof.

(D) The pharmaceutical composition as in A above, wherein the suspending agent(s) are used in amounts ranging from about 0.1 to about 0.9 mg/ml.

(E) The pharmaceutical composition as in A above, wherein the said pharmaceutical composition as physically stable as commercially available compositions in the United States of America i.e. CellCept® suspensions.

F) The pharmaceutical composition as in A above, wherein the said pharmaceutical composition is bioequivalent to commercially available compositions in the United States of America i.e. CellCept® suspensions.

(G) A process to prepare a pharmaceutical composition comprising mycophenolate mofetil in an amount of about 75 to about 200 mg/ml and a suspending agent(s) in an amount of less than 1 mg/ml, as a dry formulation suitable, when constituted with water, for forming a suspension for oral administration, where mg/ml represents concentration after constitution with water, wherein the process comprises
a. Mixing mycophenolate mofetil, suspending agent(s) and optionally anti caking agent,
b. Dissolving anti microbial agents and buffers in a suitable solvent,
c. Adding step ‘b’ to step ‘a’ and mixing till desired granulation size is obtained,
d. Drying the granulates of step ‘c’ and milling,
e. Adding sweetener, wetting agent, flavors to step ‘d’.

(H) The process as in (G) above, wherein the solvents are selected from water, ethanol and the like or mixtures thereof.

(I) A process to prepare a pharmaceutical composition comprising mycophenolate mofetil in an amount of about 75 to about 200 mg/ml and a suspending agent(s) in an amount of less than 1 mg/ml, as a dry formulation suitable, when constituted with water, for forming a suspension for oral administration, where mg/ml represents concentration after constitution with water, wherein the process comprises:

Sifting mycophenolate mofetil, suspending agent(s), sweetener, wetting agent, buffering agent, anti microbial agent, flavors, optionally anti caking agents and mixing to form a powder blend.

### DETAILED DESCRIPTION OF THE INVENTION

Before the present process and methods are described, it is to be understood that this invention is not limited to particular compounds, formulas or steps described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a compound” includes a plurality of such compounds and reference to “the step” includes reference to one or more step and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The term “physical stable” refers to negligible change in suspendibility and redispersibility of the reconstituted suspension after 1 month.

The term “dry formulation” as used herein refers to a powder blend or dry granulates.

“mg/ml” as used herein represents concentration after constitution with predetermined amount of water.

Surprisingly we have found that dry suspensions with high dose of mycophenolate mofetil can be prepared by using a suspending agent(s) in lesser amounts than those used by the prior art formulations. Also we have found that the said compositions are having comparable physical stability as the commercially available compositions in the United States of America i.e. CellCept® suspensions, in spite of using lesser amounts of suspending agent(s). The said compositions are also bioequivalent to the commercially available compositions in the United States of America i.e. CellCept® suspensions.

The present invention relates to a pharmaceutical composition comprising mycophenolate mofetil in an amount of about 75 to about 200 mg/ml and a suspending agent(s) in an amount of less than 1 mg/ml, as a dry formulation suitable, when constituted with water, for forming a suspension for oral administration, where mg/ml represents concentration after constitution with water.

The present invention relates to a process to prepare a pharmaceutical composition comprising mycophenolate mofetil in an amount of about 75 to about 200 mg/ml and a suspending agent(s) in an amount of less than 1 mg/ml, as a dry formulation suitable, when constituted with water, for forming a suspension for oral administration, where mg/ml represents concentration after constitution with water, wherein the process comprises:

a. Mixing mycophenolate mofetil, suspending agent(s) and optionally anti caking agent,

b. Dissolving anti microbial agents and buffers in a suitable solvent,

c. Adding step ‘b’ to step ‘a’ and mixing till desired granulation size is obtained,

d. Drying the granulates of step ‘c’ and milling,

e. Adding sweetener, wetting agent, flavors to step ‘d’.

The present invention also relates to a process to prepare a pharmaceutical composition comprising mycophenolate mofetil in an amount of about 75 to about 200 mg/ml and a suspending agent(s) in an amount of less than 1 mg/ml, as a dry formulation suitable, when constituted with water, for forming a suspension for oral administration, where mg/ml represents concentration after constitution with water, wherein the process comprises:

Sifting mycophenolate mofetil, suspending agent(s), sweetener, wetting agent, buffering agent, anti microbial agent, flavors, optionally anti caking agents and mixing to form a powder blend.

The composition of the invention contains high dose of mycophenolate mofetil as compared to prior art formulations. The mycophenolate mofetil may be used in the range from about 75 mg/ml to about 200 mg/ml in the composition of the present invention.

The composition of the present invention may use a single suspending agent or a mixture of two or more suspending agents. The suspending and/or viscosity increasing agent(s) used in the composition of the present invention may be selected from xanthan gum, hydroxypropyl methylcellulose, microcrystalline cellulose, colloidal silicon dioxide, sodium carboxymethylcellulose and the like or mixtures thereof. The suspending agent(s) may be used in an amount of less than 1 mg/ml, preferably from about 0.1 mg/ml to about 0.9 mg/ml.

The wetting agent used in the composition of the present invention may be selected from lecithin, preferably soy lecithin and poloxamer and the like or mixtures thereof. The wetting agent may be present in an amount ranging from about 0 to about 10 mg/ml.

The sweeteners used in the composition of the present invention may be selected from sorbitol, sorbitol powder, maltitol syrup, crystalline maltitol, sucrose, fructose, aspartame, xylitol, mannitol and the like or mixtures thereof. The dry sweeteners are preferably used for the dry granulation process of making the composition of the present invention. The sweeteners may be present in an amount ranging from about 1 mg/ml to about 600 mg/ml.

The flavors used in the composition of the present invention may be selected from mixed fruit, mint, strawberry, cherry, orange, berry and grape (optionally mixed with anise) and the like or mixtures thereof. Flavor enhancers (or bitter maskants) that may be used in the present invention include, for example Magnasweet®. The flavors may be present in an amount ranging from about 0 to about 20 mg/ml.

The buffering agents used in the composition of the present invention may be selected from citric acid, sodium citrate, sodium phosphate dibasic and the like or mixtures thereof. The buffering agents may be present in an amount ranging from about 0 to about 15 mg/ml.

The antimicrobial agents used in the composition of the present invention may be selected from methyl paraben, sodium methyl paraben, sodium benzoate, propylparaben, potassium sorbate and the like or mixtures thereof. The antimicrobial agents may be present in an amount ranging from about 0 to about 10 mg/ml.

The anti caking agents that may be used in the present invention include, but not limited to, talc, calcium silicate, magnesium trisilicate or mixtures thereof. The anti caking agents may be present in an amount ranging from about 0 to about 20 mg/ml.

The term “dry formulation” as used herein refers to a powder blend or dry granulates. The pharmaceutical compositions of the present invention may be prepared by a dry granulation method where mycophenolate mofetil, suspending agent(s), sweetener, wetting agent, buffering agent, anti microbial agent, flavors and/or anti caking agent are weighed, sifted and mixed in a suitable blender to form the powder blend. Alternatively, the dry granulates may be prepared by wet granulation method where mycophenolate mofetil and suspending agent(s) and optionally anti caking agent are mixed. Anti microbial agents and buffers are dissolved in suitable solvent selected from water, ethanol and the like or mixtures thereof and mixed with previously prepared mixture till desired granulation size is obtained. The granulates are dried and milled to reduce the particle size. Sweetener, wetting agent and flavors are added to this using suitable blender. For the purpose of the present invention, the powder blend or the dry granulates is provided in a container, such that when constituted with an appropriate volume of water ranging from about 90 ml to about 380 ml, to obtain the final predetermined volume, ranging from about 175 ml to about 470 ml.

The following examples are intended to illustrate the scope of the present invention in all its aspects but not to limit it thereto.

### EXAMPLE 1

given in Table 1

TABLE 1

| | | Quantity |
|---|---|---|
| S. No | | (mg/mL)* |
| 01 | Mycophenolate mofetil | 200 |
| 02 | Sorbitol (Neosorb P 60 W) | 500 |
| 03 | Soya lecithin powder | 3.0 |
| 04 | Xanthan gum | 0.9 |
| 05 | Citric acid anhydrous | 1 |
| 06 | Sodium citrate dihydrate | 12 |
| 07 | Aspartame | 1.5 |
| 08 | Methyl paraben | 2.8 |
| 09 | Mixed fruit flavor | 2.0 |

*Concentration after constitution with water

### Manufacturing Process:

Mycophenolate mofetil, sweetener, wetting agent, suspending agent/viscosity agent, buffering agent, anti microbial agent, flavors were weighed, sifted and mixed in a suitable blender to form powder blend.

The pharmaceutical composition of the present invention, as prepared in example 1 was subjected to physical stability studies and the results are shown in Table 2.

For the present pharmaceutical composition as prepared in example 1, measurement of the sedimentation volume and its ease of redispersion were studied for one month.

Redispersibility: It is the ease of resuspendibility, where the suspension sediment should be loosely packed such that after minimal hand shaking the sediment redisperse and reform the original suspension.

Procedure: Reconstitute the powder for suspension with required qty of water. Fixed volume of each suspension (50 ml) was kept in calibrated measuring cylinders which were stored at room temperature, one cylinder was removed at a fixed time interval and shaken to redistribute the sediment.

Suspendibility (Sedimentation Volume): The sedimentation volume is calculated using the formula Vu/Vo, where Vu is the volume of sediment and Vo is the original height of the sample.

Procedure: Reconstitute the powder for suspension with required qty of water.

The sedimentation volume were determined by keeping 50 ml of each suspensions in stoppered measuring cylinder and stored undisturbed at room temperature. The separation of clear liquid was noted at a particular time intervals. The sedimentation volume was calculated using the formula Vu/Vo, where Vu is the volume of sediment and Vo is the original height of the sample.

TABLE 2

| | | Test product | | Innovator product | |
|---|---|---|---|---|---|
| S. NO. | Test Parameters | Initial | 30 days | initial | 30 days |
| 1 | Redispersibility | Within 10 | Within 10 | Within 10 | Within 10 |
| | | sec | sec | sec | sec |
| 2 | Suspendibility | 1 | 0.88 | 1 | 0.91 |
| | (Sedimentation | | | | |
| | Volume) | | | | |

The results shown in table 2 indicate that there was no change in suspendibility and redispersibility in the composition of the present invention for a period of 1 month. It can also be seen that redispersibility & suspendibility of the composition of the present invention is comparable to that of the innovator's product.

The pharmaceutical composition as prepared in example 1 and the reference product i.e. Cellcept oral suspension were subjected to in-vivo bio-equivalence study. Two way crossover study was conducted for 48 healthy male volunteers and the plasma mycophenolic acid (MPA) concentration was measured in LCMS.

The comparative study between the formulation of the present invention and Cellcept oral suspension showed that the two formulations were bioequivalent (within 90% confidence interval for the ratios). The results are as shown in table 3.

TABLE 3

| | | Fasting | |
|---|---|---|---|
| | | 90% Confidence Interval | |
| S. NO | Parameter | Lower | Higher |
| 1. | C max | 100.64 | 115.98 |
| 2. | AUCt | 108.47 | 118.19 |
| 3. | AUC inf | 108.33 | 118.08 |

From the above bioequivalent study, it can be seen that the composition of the present invention is bioequivalent to the innovator's product.

### EXAMPLE 2

The pharmaceutical composition comprising mycophenolate mofetil may be prepared as given in Table 4.

Label Claim: Each ml contains 200 mg of mycophenolate mofetil after constitution.

TABLE 4

| Sr. | | Quantity | | |
|---|---|---|---|---|
| No. | Name of Ingredients | (mg/ml)* | % w/w | Category |
| 01 | Mycophenolate mofetil | 200.00 | 29.82 | Active |
| 02 | Sorbitol | 450.00 | 67.10 | Sweetener |
| 03 | Soy lecithin | 4.00 | 0.59 | Wetting agent |
| 04 | Xanthan gum | 0.75 | 0.11 | Suspending |
| | | | | agent(s) |
| 05 | Citric acid anhydrous | 0.55 | 0.08 | Buffering agent |
| 06 | Sodium citrate dihydrate | 10.00 | 1.49 | Buffering agent |
| 07 | Aspartame | 1.00 | 0.15 | Sweetener |
| 08 | Methyl paraben | 2.30 | 0.34 | Preservative (Anti |
| | | | | microbial agent) |
| 09 | Mixed fruit flavor | 2.00 | 0.29 | Flavor |
| | Total wt | 670.60 | | |

*Concentration after constitution with water

### Manufacturing Process:

The pharmaceutical composition was manufactured by one of the following methods

### A. Dry Granulation Method:

Mycophenolate mofetil, sweetener, wetting agent, suspending agent(s), buffering agent, anti microbial agent, flavors were weighed, sifted and mixed in a suitable blender to form powder blend.

### B. Wet Granulation Method:

Mycophenolate mofetil and suspending agent(s) were combined in a mixer. Anti microbial agents & buffers were dissolved in water & ethanol and mixed with previously prepared mixture till desired granulation size was obtained. The granulation was dried and milled to reduce the particle size. Sweetener, wetting agent and flavors were added to this using suitable blender.

The pharmaceutical composition of the present invention, as prepared in example 2 was subjected to physical stability studies and the results are shown in Table 5.

TABLE 5

| S. NO | Test | Initial | 15 days | 30 days |
|---|---|---|---|---|
| 1. | Redispersibility | Within 10 | Within 10 sec | Within 10 sec |
| | | sec | | |
| 2. | Suspendibility | 1 | 0.88 | 0.85 |
| | (Sedimentation | | | |
| | Volume) | | | |

The results shown in table 5 indicate that there was no change in suspendibility and redispersibility in the composition of the present invention for a period of 1 month.

### EXAMPLE 3

The pharmaceutical composition comprising mycophenolate mofetil may also be prepared as given in Table 6.

Label Claim: Each ml contains 200 mg of mycophenolate mofetil after constitution.

TABLE 6

| Sr. | | Quantity | | |
|---|---|---|---|---|
| No. | Name of Ingredients | (mg/ml)* | % w/w | Category |
| 01 | Mycophenolate mofetil | 200.00 | 29.38 | Active |
| 02 | Sorbitol | 450.00 | 66.11 | Sweetener |
| 03 | Soy lecithin | 4.00 | 0.59 | Wetting agent |
| 04 | Xanthan gum | 0.75 | 0.11 | Suspending agent(s) |
| 05 | Citric acid anhydrous | 0.55 | 0.08 | Buffering agent |
| 06 | Sodium citrate dihydrate | 10.00 | 1.47 | Buffering agent |
| 07 | Aspartame | 1.00 | 0.15 | Sweetener |
| 08 | Talc | 10.00 | 1.47 | Anti caking agent |
| 09 | Methyl paraben | 2.30 | 0.34 | Preservative (Anti |
| | | | | microbial agent) |
| 10 | Mixed fruit flavor | 2.00 | 0.29 | Flavor |
| | Total wt | 680.60 | | |

*Concentration after constitution with water

### Manufacturing Process:

The pharmaceutical composition was manufactured by one of the following alternative methods.

### A. Dry Granulation Method:

Mycophenolate mofetil, sweetener, wetting agent, suspending agent(s), buffering agent, anti microbial agent, anti caking agent and flavors were weighed, sifted and mixed in a suitable blender to form powder blend.

### B. Wet Granulation Method:

Mycophenolate mofetil, suspending agent(s) and anti caking agent were combined in a mixer. Anti microbial agents & buffers were dissolved in water & ethanol and mixed with previously prepared mixture till desired granulation size was obtained. The granulation was dried and milled to reduce the particle size. Sweetener, wetting agent and flavors were added to this using suitable blender.

### EXAMPLE 4

The pharmaceutical composition comprising mycophenolate mofetil may also be prepared as given in Table 7.

TABLE 7

| | | Quantity |
|---|---|---|
| S. No | Name of Ingredients | (mg/mL)* |
| 01 | Mycophenolate mofetil | 200 |
| 02 | Soya lecithin powder | 4.00 |
| 03 | Sorbitol (Neosorb P 60 W) | 400 |
| 04 | Xanthan gum | 0.3 |
| 05 | Citric acid anhydrous | 1.1 |
| 06 | Sodium citrate dihydrate | 10.0 |
| 07 | Aspartame | 1.00 |
| 08 | Methyl paraben | 2.3 |
| 09 | Mixed fruit flavor | 2.0 |
| | Total wt | 620.7 |

### Manufacturing Process.

### Wet Granulation Method

a. Soyalecithin was dissolved in warm water, granulated with mycophenolate mofetil, dried at 50° C. and sized through 60 mesh
b. Methyl paraben was dissolved in ethanol and water (1:1) and wet mixed with sorbitol, dried & sized through 60 mesh.
c. The granules of step a, step b, aspartame, citric acid, Sod. Citrate, xanthan gum, methyl paraben and flavor were mixed using suitable blender.

### EXAMPLE 5

The pharmaceutical composition comprising mycophenolate mofetil may also be prepared as given in Table 8.

TABLE 8

| | | Quantity |
|---|---|---|
| S. No | Name of Ingredients | (mg/mL)* |
| 01 | Mycophenolate mofetil | 200 |
| 02 | Soya lecithin powder | 4.00 |
| 03 | Sorbitol(Neosorb P 60 W) | 500 |
| 04 | Xanthan gum | 0.50 |
| 05 | Citric acid anhydrous | 1.1 |
| 06 | Sodium citrate dihydrate | 10.0 |
| 07 | Aspartame | 1.00 |
| 08 | Methyl paraben | 2.0 |
| 09 | Propyl paraben | 0.4 |
| 10 | color | 0.03 |
| 09 | Mixed fruit flavor | 2.0 |
| | Total Wt | 721.03 |

### A. Dry Granulation Method:

Mycophenolate mofetil, sweetener, wetting agent, suspending agent(s), buffering agent, anti microbial agent, anti caking agent and flavors were weighed, sifted and mixed in a suitable blender to form powder blend.

Although the invention has been described in terms of particular embodiments and applications, one of ordinary skill in the art, in light of this teaching, can generate additional embodiments and modifications without departing from the spirit of or exceeding the scope of the claimed invention. It should be emphasized that the above-described embodiments of the present invention, particularly any “preferred” embodiments, are merely possible examples of the invention of implementations, merely set forth for a clear understanding of the principles of the invention. Accordingly, it is to be understood that the drawings and descriptions herein are preferred by way of example to facilitate comprehension of the invention and should not be construed to limit the scope thereof.

1. A pharmaceutical composition comprising mycophenolate mofetil in an amount of about 75 to about 200 mg/ml and a suspending agent(s) in an amount of less than 1 mg/ml, as a dry formulation suitable, when constituted with water, for forming a suspension for oral administration, where mg/ml represents concentration after constitution with water.

2. The pharmaceutical composition as in claim 1, comprising mycophenolate mofetil in an amount of about 75 to about 200 mg/ml and the suspending agent(s) in an amount of less than 1 mg/ml, provided in a container marked to be filled with purified water ranging from about 90 ml to about 380 ml to obtain a final predetermined volume ranging from about 175 ml to about 470 ml.

3. The pharmaceutical composition as in claim 1, wherein the suspending agent(s) are selected from xanthan gum, hydroxypropyl methylcellulose, microcrystalline cellulose, colloidal silicon dioxide, sodium carboxymethylcellulose and the like or mixtures thereof.

4. The pharmaceutical composition as in claim 1, wherein the suspending agent(s) are used in amounts ranging from about 0.1 to about 0.9 mg/ml.

5. The pharmaceutical composition as in claim 1, wherein the said pharmaceutical composition is as physically stable as the commercially available compositions in the United States of America i.e. CellCept® suspensions.

6. The pharmaceutical composition as in claim 1, wherein the said pharmaceutical composition is bioequivalent to the commercially available compositions in the United States of America i.e. CellCept® suspensions.

7. A process to prepare a pharmaceutical composition comprising mycophenolate mofetil in an amount of about 75 to about 200 mg/ml and a suspending agent(s) in an amount of less than 1 mg/ml, as a dry formulation suitable, when constituted with water, for forming a suspension for oral administration, where mg/ml represents concentration after constitution with water, wherein the process comprises:
a. Mixing mycophenolate mofetil, suspending agent(s) and optionally anti caking agent,
b. Dissolving anti microbial agents and buffers in a suitable solvent,
c. Adding step ‘b’ to step ‘a’ and mixing till desired granulation size is obtained,
d. Drying the granulates of step ‘c’ and milling,
e. Adding sweetener, wetting agent, flavors to step ‘d’.

8. The process as in claim 7, wherein the solvents are selected from water, ethanol and the like or mixtures thereof.

9. A process to prepare a pharmaceutical composition comprising mycophenolate mofetil in an amount of about 75 to about 200 mg/ml and a suspending agent(s) in an amount of less than 1 mg/ml, as a dry formulation suitable, when constituted with water, for forming a suspension for oral administration, where mg/ml represents concentration after constitution with water, wherein the process comprises:
Sifting mycophenolate mofetil, suspending agent(s), sweetener, wetting agent, buffering agent, anti microbial agent, flavors, optionally anti caking agents and mixing to form a powder blend.
